(19) United States
(12) Patent Application Publication
(10) Pub. No.: US 2026/0295257 A1
(43) Pub. Date: Oct. 1, 2026

(54) RETINA IMAGE FORMATION ENHANCEMENT THROUGH ACTIVE NANOPARTICLE-BASED PHOTORECEPTORS

(71) Applicant: Purdue Research Foundation, West Lafayette, IN (US)

(72) Inventor: Afshin Izadian, Carmel, IN (US)

(73) Assignee: Purdue Research Foundation, West Lafayette, IN (US)

(21) Appl. No.: 19/578,666

(22) Filed: Mar. 25, 2026

Related U.S. Application Data
(60) Provisional application No. 63/779,395, filed on Mar. 28, 2025.

(51) Int. Cl. A61N 1/36; B82Y 5/00; B82Y 20/00; G06T 7/13

(52) U.S. Cl. CPC A61N 1/36046; B82Y 5/00; B82Y 20/00; G06T 7/13; G06T 2207/30041

(57) ABSTRACT

An artificial photoreceptors (APR) includes a nano-dielectric core, a plurality of optical dipoles each optical dipole attached to the surface of the nano-dielectric core, and each optical dipole includes a first nano-particle configured to generate a positive voltage when receiving a light pulse having a predetermined wavelength, and a second nano-particle configured to generate a negative voltage when receiving a light pulse having the predetermined wavelength, wherein i) a plurality of a first set of the first and second nano-particles of the plurality of optical dipoles cancel each other's voltages such that a plurality of APRs only generate voltages at locations corresponding to edge-detected signals representing edges of an object; or ii) a plurality of a second set of the first and second nano-particles of the plurality of optical dipoles generate positive and negative voltages to sequentially generate responses for biological receptors.

### CROSS-REFERENCE TO RELATED APPLICATIONS

The present non-provisional patent application is related to and claims the priority benefit of U.S. Provisional Patent Application 63/779,395, filed Mar. 28, 2025, the contents of which are hereby incorporated by reference in its entirety into the present disclosure.

### STATEMENT REGARDING GOVERNMENT FUNDING

None.

### TECHNICAL FIELD

The present disclosure generally relates to vision systems and in particular to artificial photoreceptors (ARs) and the accompanying vision system.

### BACKGROUND

This section introduces aspects that may help facilitate a better understanding of the disclosure. Accordingly, these statements are to be read in this light and are not to be understood as admissions about what is or is not prior art.

Total blindness or partial blindness affects large swath of population. There are myriad reasons for blindness, however, retinal diseases are amongst one of the primary causes. Retina is a multi-layer tissue in the back of the eye which includes rod and cone photoreceptors (PRs) that are adapted to convert images focused thereon by the optical lens of the eye into electrical signals that are conveyed by the optical nerves to the brain where images are perceived. Loss of PRs due to disease or accidents can cause partial or total blindness. Photoreceptors detect light and stimulate downstream neurons in the retina. The number of individuals affected by partial and total blindness is staggering. In 2010, the number of people suffering from such ailment was around 1 million people in the United States alone suffering profound vision loss, with another 2.4 million having some degree of visual impairment (see US Pub. App. 20140128972 for Khraiche et al.). Currently about 7 million people suffer from some level of blindness. One approach to addressing retinal issues is to place artificial photoreceptors by or near the retina. While part of retina may be damaged or diseased, a number of PRs may retain sufficient health that can be used to process information. By stimulating the healthy PRs, it may be possible to regain some vision for the affected individuals.

Currently, the use of artificial photoreceptors typically requires image capture by a camera and processing of images onto nanodevices that stimulate healthy PRs. However, resolution of a such images and the information associated with the images may be too much for real-time processing. Additionally, such artificial photoreceptors are easily saturated resulting in a perceived whitewashed image.

Therefore, there is an unmet need for a novel artificial photoreceptor (AR) and a system that operates such ARs which can effectively process images in real-time for individuals with partial or total blindness.

### SUMMARY

A vision system is disclosed. The vision system includes an image capture system which includes an image capture device having an image sensing element configured to receive light from an object and generate object-shape signals commensurate with shape of the object a processor executing software maintained on a non-transitory memory. The processor is configured to receive the object-shape signals, perform edge detection on the object-shape signals, generate edge-detected signals representing edges of the object. The visions system further includes one or more light-generating devices, each configured to generate pulses of light at a predetermined transmitted wavelength; a plurality of artificial photoreceptors (APRs). Each APR includes a nano-dielectric core, a plurality of optical dipoles each optical dipole attached to the surface of the nano-dielectric core. Each optical dipole includes a first nano-particle configured to generate a positive voltage when receiving a light having a predetermined wavelength, and a second nano-particle configured to generate a negative voltage when receiving a light having the predetermined wavelength. One or more dielectric coatings applied to the nano-dielectric core and each of the plurality of optical dipoles. The processor controls the one or more light generating devices based on the edge-detected signals such that: i) a plurality of a first set of the first and second nano-particles of the plurality of optical dipoles cancel each other's voltages such that the plurality of APRs only generate voltages at locations corresponding to the edge-detected signals; or ii) a plurality of a second set of the first and second nano-particles of the plurality of optical dipoles generate positive and negative voltages to sequentially generate responses for biological receptors.

In the above vision system, the first nanoparticle of each of the plurality of optical dipoles is a gold nanoparticle.

In the above vision system, the second nanoparticle of each of the plurality of optical dipoles is a silver nanoparticle.

In the above vision system, the nano-dielectric core of each of the plurality of optical dipoles is made of a material having a dielectric constant above a dielectric threshold.

In the above vision system, the nano-dielectric core of each of the plurality of dipoles is made of BaTiO₂ or Graphene.

In the above vision system, each of the one or more dielectric coatings is made of a plasmon dielectric material.

In the above vision system, each of the one or more dielectric coatings is made of a material selected from the group consisting of polyvinylidene fluoride (PVDF), polytetrafluoroethylene (PTFE), fluorinated ethylene propylene (FEP), polyvinyl chloride (PVC), chlorinated polyvinyl chloride (CPVC), high-density polyethylene (HDPE), and combinations thereof.

In the above vision system, object-shape signals include pixel intensity values for a plurality of pixels.

In the above vision system, the processor performs edge detection based on abrupt changes in pixel intensity of the plurality of pixels.

In the above vision system, the processor performs edge detection based on one or more of object recognition, segmentation, feature extraction, or combinations thereof.

An artificial photoreceptors (APR) is also disclosed. The APR includes a nano-dielectric core, a plurality of optical dipoles each optical dipole attached to the surface of the nano-dielectric core. Each optical dipole includes a first nano-particle configured to generate a positive voltage when receiving a light pulse having a predetermined wavelength, and a second nano-particle configured to generate a negative voltage when receiving a light pulse having the predetermined wavelength. One or more dielectric coatings is applied to the nano-dielectric core and each of the plurality of optical dipoles. A plurality of a first set of the first and second nano-particles of the plurality of optical dipoles cancel each other's voltages such that a plurality of APRs only generate voltages at locations corresponding to edge-detected signals representing edges of an object. Or a plurality of a second set of the first and second nano-particles of the plurality of optical dipoles generate positive and negative voltages to sequentially generate responses for biological receptors.

In the above APR, the first nanoparticle of each of the plurality of optical dipoles is a gold nanoparticle.

In the above APR, the second nanoparticle of each of the plurality of optical dipoles is a silver nanoparticle.

In the above APR, the nano-dielectric core of each of the plurality of optical dipoles is made of a material having a dielectric constant above a dielectric threshold.

In the above APR, the nano-dielectric core of each of the plurality of dipoles is made of BaTiO₂ or Graphene.

In the above APR, each of the one or more dielectric coatings is made of a plasmon dielectric material.

In the above APR, each of the one or more dielectric coatings is made of a material selected from the group consisting of polyvinylidene fluoride (PVDF), polytetrafluoroethylene (PTFE), fluorinated ethylene propylene (FEP), polyvinyl chloride (PVC), chlorinated polyvinyl chloride (CPVC), high-density polyethylene (HDPE), and combinations thereof.

In the above APR, the edge-detected signals include pixel intensity values for a plurality of pixels.

In the above APR, edge detection of the object is based on abrupt changes in pixel intensity of the plurality of pixels.

In the above APR, edge detection of the object is based on one or more of object recognition, segmentation, feature extraction, or combinations thereof.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 is an example schematic of an Artificial Photoreceptors (APR), according to the present disclosure.

FIGS. 2A-2F are images of a cross in different configuration provided to show edge detection, according to the present disclosure.

FIG. 3 is a synthesis schematic for making an Artificial Photoreceptor (APR) according to the present disclosure according to one embodiment.

FIG. 4A is a schematic of a single core APR attached to a nano-core, where single-core APRs use one type of metallic core in their structure as shown in FIG. 4A.

FIG. 4Bb is a schematic of a dipole core APR attached to a nano-core, where multi-core APRs are a combination of two separately processed Au and Ag nano-cores that can generate positive and negative voltages respectively as shown in FIG. 4B.

FIG. 4C is a schematic of BaTiO₂ nano-core with a carrier surface functionalized to cover many of single core APRs.

FIG. 4D is a scanning electron microscope image displaying a prototype of the system shown in FIG. 4C.

FIGS. 5A-5D provide fluorescent images that show APRs simulated at 550 nm, the charges generated by APRs triggered the dye to emit a 635 nm wavelength, wherein FIG. 5A shows the control for gold-based APR with no response, FIG. 5B shows the response of gold nano particular (AuNP)-based APRs, FIG. 5C shows the control for the silver-based APR, and FIG. 5D shows the response of AgNP-based APRs.

FIG. 6A shows APRs were intravitreally injected into the rd1 PDE mice eye.

FIG. 6B shows the pupillary response rescued post-APR intravitreal injection.

FIG. 6C shows the pupillary response with no treatment.

FIGS. 7A and 7B are Fundus images showing a retina (FIG. 7A: prior to APR injection) and FIG. 7B: day 1 post injection).

FIGS. 7C and 7D are optical coherence tomography (OCT) images of the retina (FIG. 7C: day 1 post injection, and FIG. 7D: day 3 post injection) providing images at different vantage points.

FIGS. 7E, 7F, and 7I provide graphs of voltage in μv vs. time in milliseconds showing electrical activity from retina in response to various stimuli, where a relatively flat responses shows lack of response, while a negative peak followed by a positive peak represent healthy retinal response, but a solo positive peak represent APR responses, FIGS. 7E and 7I represent no-response (blind mice), and FIG. 7F represents APR responses (positive peak) followed by flat response indicating APRs stimulating retina.

FIGS. 7G and 7H provide a Fundus and an OCT images showing the retina (FIG. 7G: Fundus image day 7 post injection and the OCT image of the retina FIG. 7H: day 7 post injection).

FIG. 8 is complex graph of wavelength in nm vs. particle diameter in nm and vs. potential in V.

FIG. 9A is a graph of normalized growth vs. AuNP concentration in μg/mL, ppm which provide results of proliferation and cell apoptosis assays on different concentrations of gold nanoparticles on human retinal endothelial vascular cells (HREC cells).

FIGS. 9B and 9C provide bar graphs of vascular density in pixel area vs. AuNP intravitreal injection in μg/ml for superficial (FIG. 9B) and Deep (FIG. 9C) in C56BL/6 mice with different concentrations which show the superficial and deep retinal vasculature by immunohistochemistry 14 days following injection.

FIG. 10A shows the structure of a multi-core APRs used in these experiments and the recorded RGC spike rate results. FIG. 10A shows the multi-core of AuNP and AgNP, paired and attached to the BaTiO2 carrier where the APR's outer shell is not coated (e.g., with PVDF).

FIG. 10B shows a similar APR to FIG. 10A, but the outer shell is covered with PVDF and is referred to herein as multi-core APR-coated.

FIG. 10C shows the spike rate generated while multi-core APR was used. It shows that the dark, green (550 nm), and blue (470 nm) individually did not excite these APRs.

FIG. 10D shows the results of utilizing multi-core APR-coated.

FIGS. 10E and 10F show the spike rate of multi-core APR and multi-core APR-coated to the combined green and blue wavelengths at various light intensities.

FIG. 11 is a block diagram of the vision system according to one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure, the term “about” can allow for a degree of variability in a value or range, for example, within 15%, within 10%, within 5%, or within 1% of a stated value or of a stated limit of a range.

In the present disclosure, the term “substantially” can allow for a degree of variability in a value or range, for example, within 85%, within 90%, within 95%, or within 99% of a stated value or of a stated limit of a range.

A novel artificial photoreceptor (AR) and a system that operates such ARs are disclosed which can effectively process images in real-time for individuals with partial or total blindness. Towards this end a novel AR is disclosed which along with the disclosed vision system can provide images of edges of objects rather than complete object detail for faster and real-time processing by the system, the ARs, and the remaining healthy photoreceptors in the retina.

Nanotechnology-based Artificial Photoreceptors (APRs) are innovative light-responsive structures that convert specific wavelengths to action potentials that trigger retina ganglion cells (RGCs). These APRs aim to replace damaged photoreceptors to address total or partial blindness. However, treating diseases like macular degeneration and retinitis pigmentosa requires more advanced prosthetics with complicated functionalities. So far, these prosthetics have reached limited electrode density and resolution, but no functional vision has been restored. APRs are highly sensitive to light intensities and are easily saturated, thus generating a whitewashed image on the retina. Limited functionalities prevent APRs from mimicking the natural response of photoreceptors. APRs also act as a single-point perception, which further limits the image resolution enhancement. Hence, the image projected by these prosthetics is not recognizable by the brain. Therefore, there is a disconnect between the ability to remotely, precisely, and efficiently excite individual APRs and create a recognizable image in the brain. Imprinting a pulsed high-resolution outline of an object on the retina is an effective vision restoration technique that provides recognizable details of an image. However, such a technique requires advanced APR structures to generate controlled action potentials only on the pulsed outlines. This present disclosure provides a way to enhance high-resolution vision by enabling full control over the operation of individual APRs in connection with a companion vision system to generate outlines of objects for more effective object recognition by the brain. In short, the present disclosure provides a system which aims to 1) excite RGCs on demand with controllable multi-core APRs and 2) increase the spatial resolution of objects.

To achieve these goals, an APR with an optical dipoles is disclosed herein. Utilizing a pair of cationic and anionic plasmonic materials can enable the on-off, on-demand operation of multi-core APRs. Each APR includes a nano-dielectric core and a plurality of optical dipoles. Each optical dipole is attached to the surface of the nano-dielectric core. Each optical dipole includes a first nano-particle configured to generate a positive voltage when receiving a light having a predetermined wavelength, and a second nano-particle configured to generate a negative voltage when receiving a light having the predetermined wavelength. One or more dielectric coatings are applied to each of the nano-dielectric cores and each of the plurality of optical dipoles. These APRs operate such that i) a plurality of a first set of the first and second nano-particles of the plurality of optical dipoles cancel each other's voltages such that the plurality of APRs only generate voltages at locations corresponding to edge-detected signals provided by the vision system; or ii) a plurality of a second set of the first and second nano-particles of the plurality of optical dipoles generate positive and negative voltages to sequentially generate responses for biological receptors.

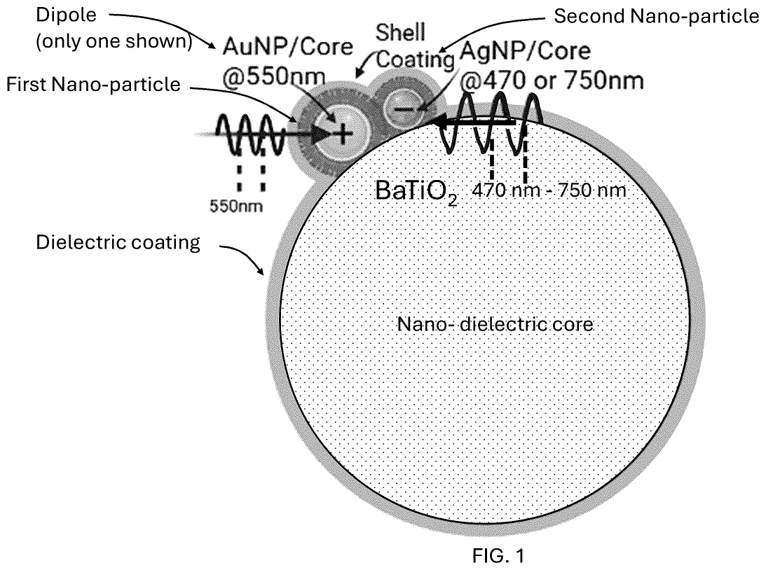

Referring to FIG. 1, an example schematic of the APR according to the present disclosure is provided. By incorporating the gold and silver nanoparticles in APRs and mounting them on a BaTiO₂ carrier, as shown in FIG. 1, a careful selection of wavelength excitations can control the opposite voltage amplitudes from AuNP and AgNP. These two voltages can cancel each other at a suitable excitation and cause the overall APR excitation to fall under the action potential, hence turning the APR “off.” Therefore, by energizing the device shown in FIG. 1 with the selected wavelength, one can excite everywhere except the edges, thus allowing only devices associated with the edges to cause excitation of the ganglion cells.

According to one embodiment, the first nanoparticle of each of the plurality of optical dipoles is a gold nanoparticle.

According to one embodiment, the second nanoparticle of each of the plurality of optical dipoles is a silver nanoparticle.

According to one embodiment, the nano-dielectric core of each of the plurality of optical dipoles is made of a material having a dielectric constant above a dielectric threshold.

According to one embodiment, the nano-dielectric core of each of the plurality of dipoles is made of BaTiO₂ or Graphene.

According to one embodiment, each of the one or more dielectric coatings is made of a plasmon dielectric material.

According to one embodiment, each of the one or more dielectric coatings is made of a material selected from the group consisting of polyvinylidene fluoride (PVDF), polytetrafluoroethylene (PTFE), fluorinated ethylene propylene (FEP), polyvinyl chloride (PVC), chlorinated polyvinyl chloride (CPVC), high-density polyethylene (HDPE), and combinations thereof.

The vision system includes an image capture system such as a camera, e.g., a high-definition camera, which includes an image sensing element, e.g., a charge-coupled device (CCD) that is configured to receive light from an object and generate object-shape signals commensurate with shape of the object. The vision system may also include a processor that is executing software maintained on a non-transitory memory and which is configured to i) receive the object-shape signals, ii) perform edge detection on the object-shape signals, and iii) generate edge-detected signals representing edges of the object. The vision system may further include one or more light-generating devices, each configured to selectively generate light at a predetermined transmitted wavelength. When light at the predetermined wavelength is shone on the APRs, the APRs, as discussed above, respond by either generating voltages that are cancelled by the optical dipoles, or generate signals that can stimulate photoreceptors. Thus, where the dipoles cancel each other's voltages, no signal is generated by the APRs, but where signals are generated, these signals are transmitted to the healthy photoreceptors for recognizing edges of the object. Increased outline resolution and advanced APR optical excitation methods at the desired ganglion cell locations will improve the spatial resolution of the image projected on the retina.

There are many edge detection algorithms that can be used by the vision system. These include algorithms that operate based on abrupt changes in pixel intensity of an object to determine edges of the object. Other edge detection algorithms include edge detection based on one or more of object recognition, segmentation, feature extraction, or combinations thereof. One such edge detection algorithm is Canny edge detection algorithm that can be used to identify objects. This algorithm identifies edges by using a multi-stage approach that includes noise reduction, gradient calculation (essentially, to identify edges, the intensity gradients of the image are calculated using an operator, e.g., a Soble operator which calculates the gradient in both the x and y directions, resulting in a magnitude and direction for each pixel), non-maximum suppression (essentially, provides thin, single-pixel edges, whereby non-maximum suppression identifies local maxima in the gradient magnitude image by comparing the gradient magnitude of a pixel with its neighbors along the gradient direction, suppressing pixels that are not local maxima), and hysteresis thresholding (essentially, by determining which edges are real and which are spurious, hysteresis thresholding uses threshold values to detect edges). Noise reduction is accomplished by applying a filter, e.g., a Gaussian filter to the image (essentially, calculates a weighted average of neighboring pixels, with closer pixels to a center having progressively higher weights), smoothing the image to thus facilitate edge detection of the image.

The ability to control the excitation of APRs at high densities will improve the quality of vision restoration. The goal is to improve APR response to incoming light wavelengths to prevent saturation and respond to turn-off commands. The new cores added to the APRs will convert them to pair-electrode excitation hubs that resemble a two-point perception needed for enhanced resolution. Our turn-off mechanism involves silver nanoparticles (AgNPs) that, when excited, generate an opposite polarity voltage and can neutralize excitations by gold nanoparticles (AuNPs). The strength of this approach is that a turn-off signal is a beam of light that travels to the desired pixels and simply turns them off. This approach imprints high-resolution excitation areas on the retina, allowing for high spatial resolutions (i.e., high resolution of edges vs. a whitewashed image of object).

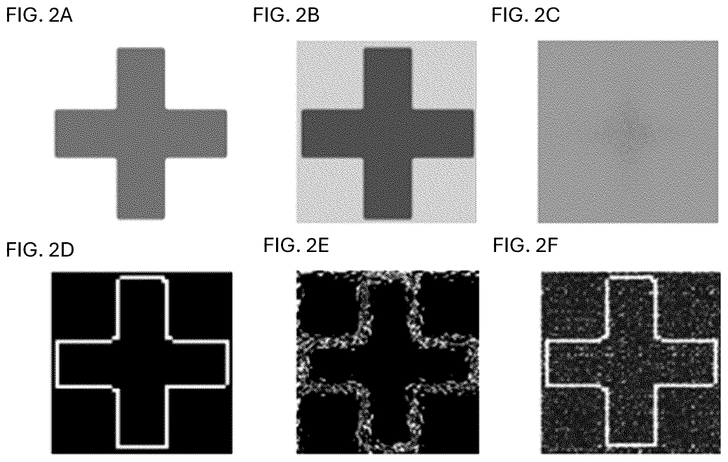

Referring to FIGS. 2A-2F, edge detection advantage is provided. FIG. 2A is an image of a cross as the image must pass a mono-color filter, forming a green-toned image (FIG. 2B). This image on the retina does not provide a significant difference for APR stimulation and was considered intense enough to whitewash the entire region projecting an image shown in FIG. 2C. However, utilizing advanced image processing and edge detection tools such as Canny edge detection algorithm discussed above, one can distinguish the areas that APRs must be off and the areas that APRs must be on. The original image (FIG. 2A), after passing through the Canny filter, provides the outlines in bright color (APR on) and the background in dark color (APR off) shown in FIG. 2D. Considering that the proposed multi-core APRs can respond to the on-off commands of FIG. 2D, the projected areas on the retina will match the shape of the object. The image's resolution will depend on the density of APRs (low-density FIG. 2E, and high-density FIG. 2F).

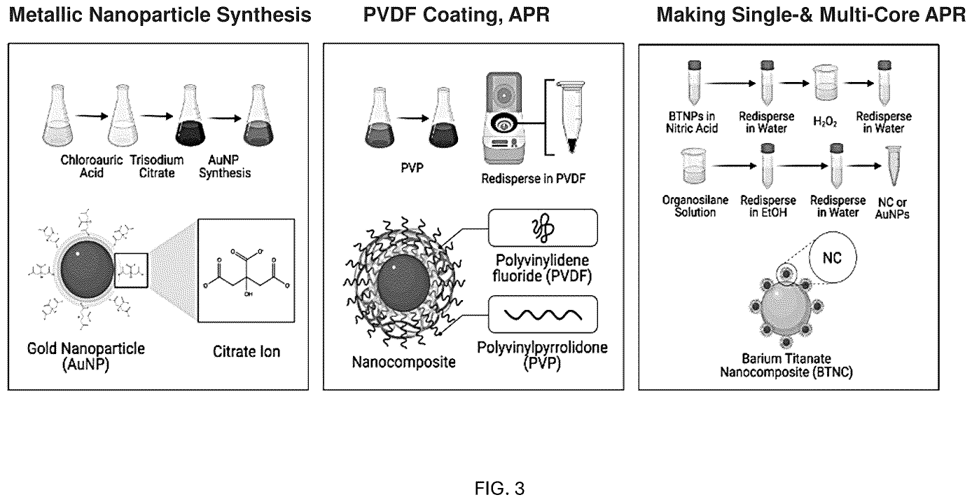

Referring to FIG. 3, a synthesis schematic for making the APR according to the present disclosure is provided according to one embodiment. 20 mg of HAuCl₄ (Sigma-Aldrich) was suspended in 0.7 mL Milli-Q water and carefully added to 195 mL Milli-Q boiling water. 30 mL of 38 mM trisodium citrate was added dropwise to the boiling water. While the temperature was kept constant, the solution was stirred vigorously. After adding trisodium citrate solution, the color gradually changed from light yellow to dark purple and eventually to a wine red, showing the formation of nanoparticles and their growth rate. The timing of citrate exposure and its concentration were used to determine the desired size of nanoparticles. Transmission electron microscopy (TEM) and Dynamic Light Scattering (DLS) showed the quality of the nanoparticles. FIG. 3 show the process of making nanoparticles, coating and mounting in solution. The metallic core nanoparticles were further processed with a plasmon PVDF-HFP coating and mounted on a high-dielectric BaTiO₂ carrier. This process requires a linker between the metallic nanoparticle and the PVDF.

Polyvinylpyrrolidone (PVP) was added to the synthesized metallic nanoparticles prior to removing them from heat. This replaced the citrate coating of the nanoparticles, serving as a linker molecule between metallic nanoparticles and the outer shell polymer. Poly(vinylidene fluoride-co-hexafluoropropylene) (PVDF-HFP) as a plasmon biosafe dielectric polymer coats the mixture of PVP and metallic nanoparticles. The nanoparticle solution and PVDF were separately dissolved in dimethylformamide (DMF) and then mixed to form the desired outer shell and nano-cores. PVP in this structure is not water-soluble as the APRs are firmly bonded to metal nanoparticles; the APRs are at fairly low temperatures to be affected by water and are coated with PVDF. The final size of the PVP-PVDF coated nanoparticles i.e., nano-cores makes them ideal to stay in solution form.

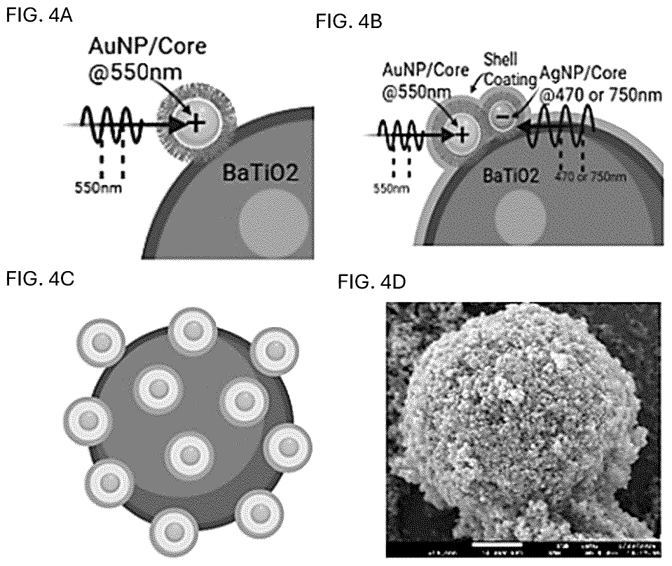

Referring back to FIG. 1, for sake of simplicity, only one dipole is shown. However, the nano-core is attached to a large number of such dipoles. Single-core Artificial Photoreceptors (APRs) use one type of metallic core in their structure as shown in FIG. 4A. Multi-core APRs are a combination of two separately processed Au and Ag nano-cores that can generate positive and negative voltages respectively as shown in FIG. 4B. The BaTiO₂ carrier surface will be functionalized to cover many of these nano-cores, as shown in FIG. 4C, with a prototype shown in FIG. 4D.

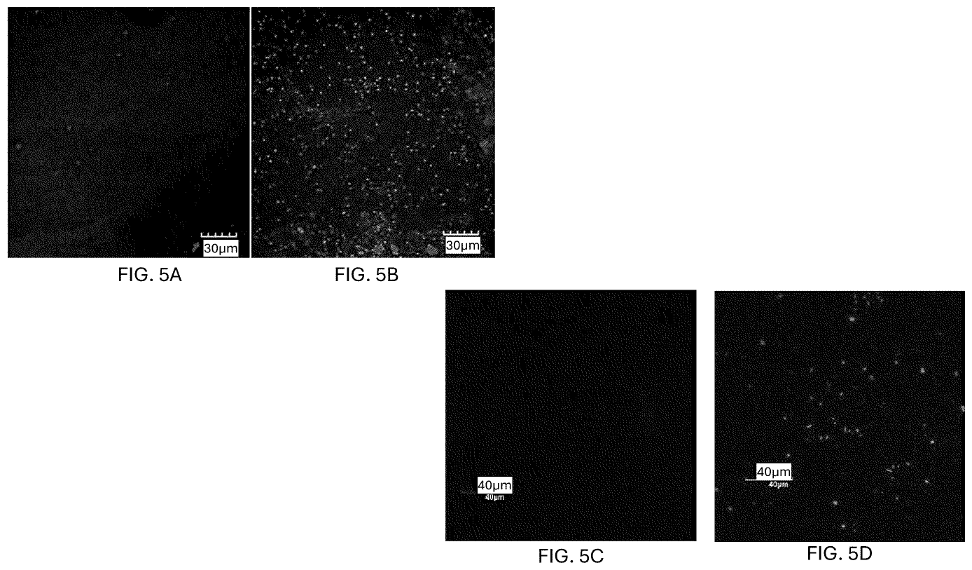

Di-8-ANEPPS (Thermofisher Scientific®) was utilized to image the electric charges generated by APRs of the present disclosure. Upon stimulating APRs at 550 nm, the charges generated by APRs triggered the dye to emit a 635 nm wavelength, as shown in FIGS. 5A-5D. These figures show a uniform distribution of the APRs in the solution and their ability to produce charges. FIG. 5A shows the control for gold-based APR with no response. FIG. 5B shows the response of AuNP-based APRs. FIG. 5C shows the control for the silver-based APR. FIG. 5D shows the response of AgNP-based APRs.

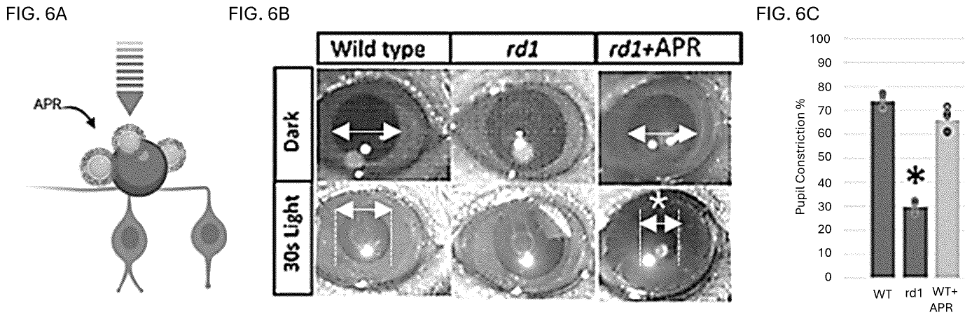

Mice pupillary light reflex (PLR) measurements were performed which followed a standardized protocol. To avoid measurement variability due to anxiety-induced responses, mice were lightly anesthetized with ketamine and xylazine and placed on a platform in a dark room. PLR recordings are made using an infrared video camera while 68 lux white light (with low green 550 nm content) from a halogen bulb was exposed in the other eye through a light guide connected to the lamp source. Neutral density filters are used to cover a range of irradiance levels. Each stimulation was presented for 30 seconds. ImageJ software analyzed the video frames, marked the pupil boundary using the circle tool, and computed the area within the boundary. APRs shown in FIG. 6A were intravitreally injected into the rd1 PDE mice eye. The pupillary response was rescued post-APR (see FIG. 6B) intravitreal compared to poor response with no treatment (see FIG. 6C). This showed that our injected APRs could interface and excite ganglion cells and send signals to the brain.

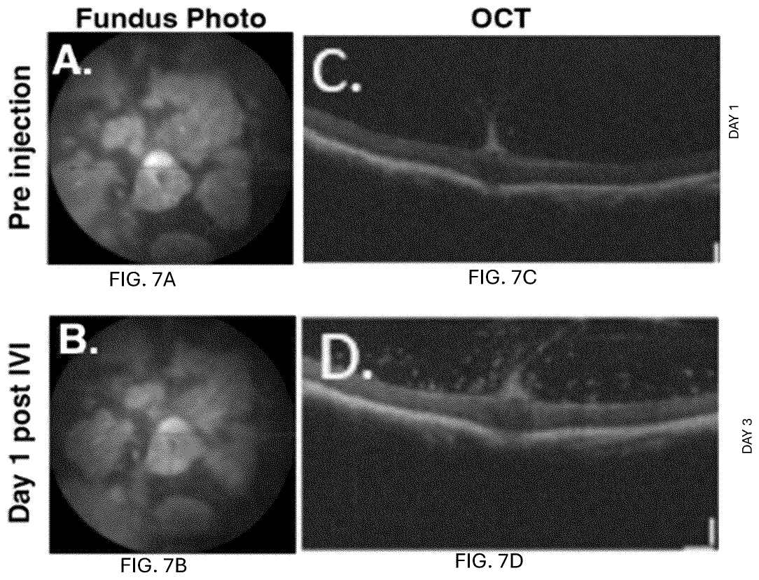

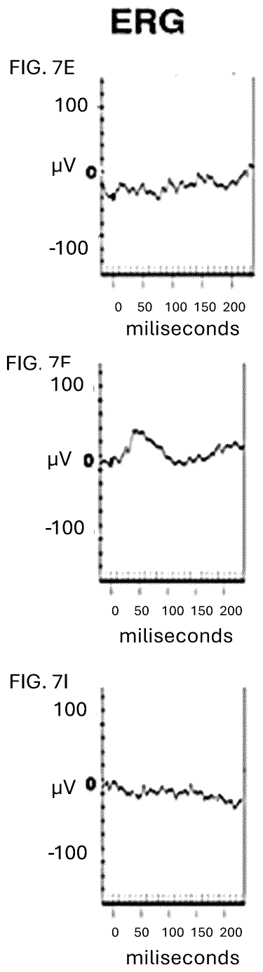

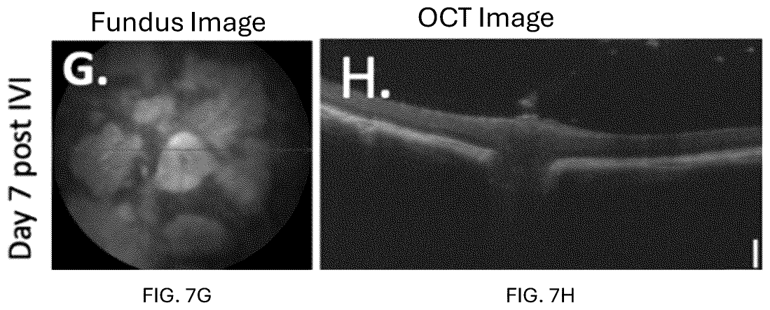

To detect the presence of APRs in the eye, we performed longitudinal optical coherence tomography (OCT) imaging of mice following intravitreal injections. We observed a significant presence of nanocomposite at the retina's surface and inside the vitreous cavity on days 1 and 3 post-intravitreal injections. Referring to FIGS. 7A and 7B, Fundus images are provided showing a retina, FIG. 7A: prior to APR injection; and FIG. 7B: day 1 post injection.

Referring to FIGS. 7C and 7D optical coherence tomography (OCT) images are provided of the retina, FIG. 7C: day 1 post injection; and FIG. 7D: day 3 post injection, providing images at different vantage points.

Referring to FIGS. 7E, 7F, and 7I, graphs of voltage in μv vs. time in milliseconds showing electrical activity from retina in response to various stimuli, where a relatively flat responses shows lack of response, while a negative peak followed by a positive peak represent healthy retinal response, but a solo positive peak represent APR responses; FIGS. 7E and 7I represent no-response (blind mice), and FIG. 7F represents APR responses (positive peak) followed by flat response indicating APRs stimulating retina.

Electroretinography (ERG) was performed following intravitreal injection of APRs to study the ability of the construct to generate a retinal response. Pre-injection (FIGS. 7A, 7C, and 7E), one-day post-APR injection (FIGS. 7B, 7D, and 7F), and 7 days post-injection (FIGS. 7G, 7H, and 7I). Interestingly, there is no wave on ERG, which shows a lack of photoreceptors. Referring to FIGS. 7G and 7H a Fundus and an OCT images are provided showing the retina (FIG. 7G: Fundus image day 7 post injection and the OCT image of the retina FIG. 7H: day 7 post injection).

Our artificial photoreceptors induced ERG response on the first-day post intravitreal injection in rd1 mice (FIG. 7F), which shows a flat a-wave response, indicating the lack of natural photoreceptors. The retinal electrical activity lasted until nanoparticles were no longer detectable by OCT. Following clearance of nanocomposite from the vitreous cavity, depicted by the absence of hyper-reflective spots on OCTs, the ERG became flat in rd1 mice (FIG. 7I).

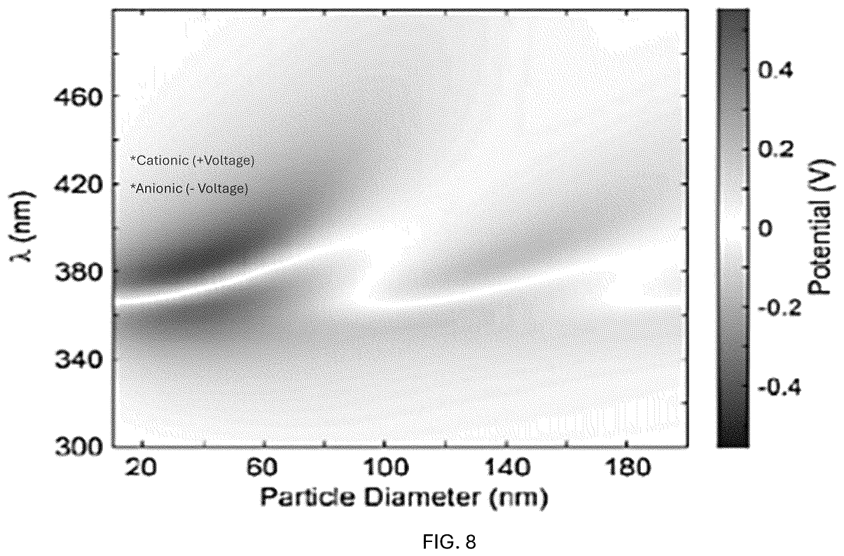

Silver Nanoparticles exhibit a wide range of absorption wavelengths, reaching 350 nm as nanospheres and 800-1200 nm as nanorods (as shown in FIG. 8). At specific operating points, shapes, and sizes, these nanoparticles also induce negative potential. FIG. 8 shows the negative potential from Ag Nanospheres when excited by wavelengths close to 350 nm to maximize the opposite voltage amplitude. This negative voltage proves that AgNPs can neutralize the effect of AuNPs by shortening the circuit between the AgNP (negative potential), AuNP (positive potential). This cancellation will reduce the excitation voltage at the ganglion cells and switch APRs off. When the AgNP is not stimulated, the AuNP will induce the voltage to excite the RGCs.

Titanium Dioxide has extraordinary light absorption capabilities and can enhance the charge separation in the plasmonic process. However, it does not provide plasmonic characteristics unless it is combined with Au or Ag nanoparticles. Thus, according to one embodiment, use of Au/TiO₂ and Ag/TiO₂ enhances their operation when the off trigger is needed. Due to the superior characteristics of the TiO₂ combination with these cores, their overall action potential shall be enhanced significantly. It should be noted that these cores are mounted as a pair on the BaTiO₂ nanoceramics. Adding TiO₂ to the cores of multi-core APRs shall enhance their operation.

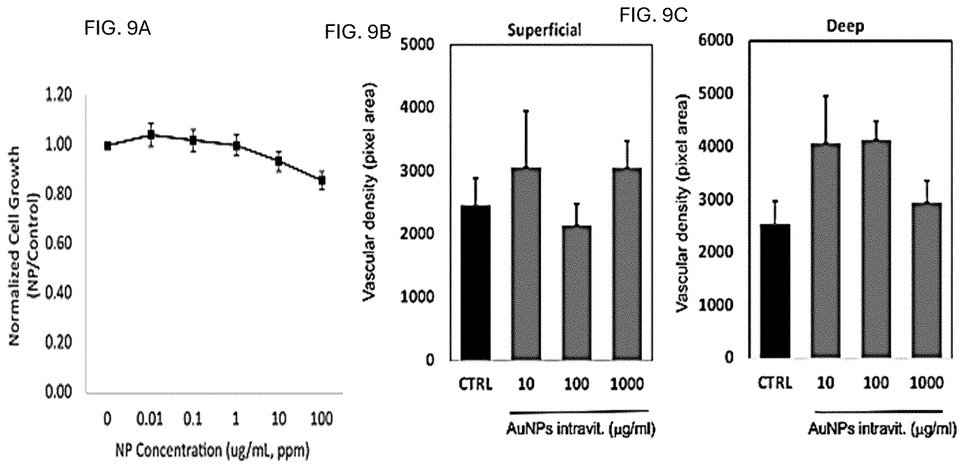

To analyze the toxicity of cores, we have performed a series of tests to identify side effects on the potential release of AuNPs in nanospheres from the device to the vitreous cavity or direct cell contact toxicity. One of the concerns is the effect on blood vessels. We have performed proliferation and cell apoptosis assays on different concentrations of gold nanoparticles (see FIG. 9A) on human retinal endothelial vascular cells (HREC cells). There were no differences in proliferation assay between the groups. We also tested the intravitreal injection of AuNPs in C56BL/6 mice with different concentrations and evaluated the superficial and deep retinal vasculature by immunohistochemistry 14 days following injection. There were no significant changes in superficial or deep retinal vasculature with different concentrations of AuNPs (shown in FIG. 9B and FIG. 9C).

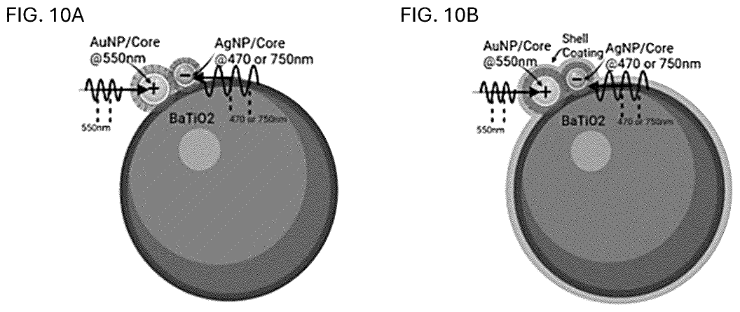

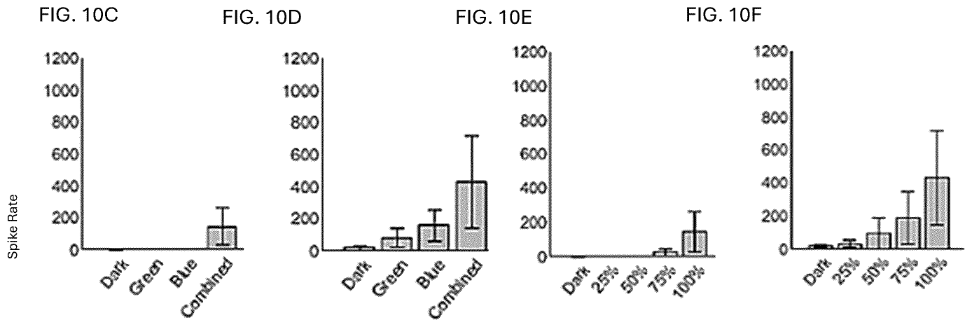

In another experiment, the prototype of multi-core APRs was made and cocultured with human retina ganglion stem cells. The mixture was placed in the MEA Axon and exposed to various light conditions. FIGS. 10A-10F are related to this experiment. FIG. 10A shows the structure of the multi-core APRs used in these experiments and the recorded RGC spike rate results. FIG. 10A shows the multi-core of AuNP and AgNP, paired and attached to the BaTiO2 carrier where the APR's outer shell is not coated with PVDF. FIG. 10B shows a similar APR to FIG. 10A, but the outer shell is covered with PVDF and is called multi-core APR-coated. FIG. 10C shows the spike rate generated while multi-core APR was used. It shows that the dark, green (550 nm), and blue (470 nm) individually did not excite these APRs. A high ganglion cell activity was observed under excitation of combined green & blue wavelengths. The results of utilizing multi-core APR-coated is shown in FIG. 10D. The outer shell resulted in a higher spike rate from the combined green and blue stimulations. However, the APRs also responded to individual stimulations of green or blue by increasing the RGC spike rates. The response to blue stimulated more RGCs, which confirms higher voltage excitations from AgNP cores than AuNP cores. FIGS. 10E and 10F show the spike rate of multi-core APR and multi-core APR-coated to the combined green and blue wavelengths at various light intensities.

The structural advancement of our multi-core APR prototype according to the present disclosure successfully prevented light intensity saturations (FIG. 10E). It also demonstrated an advanced functionality of turning APR and RGC excitation off on demand (FIG. 10C). The outer PFVD coating made the multi-core APR-coated prototype responses stronger (FIG. 10F).

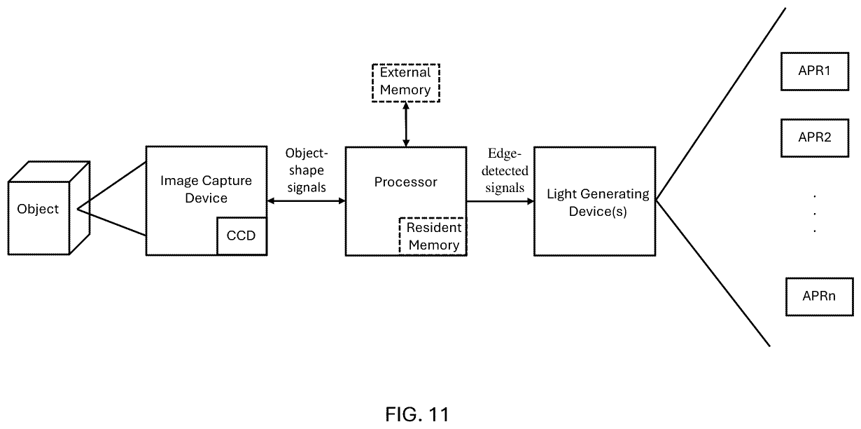

Referring to FIG. 11, a block diagram of the vision system according to one embodiment of the present disclosure is shown. As discussed above, the vision system includes an image capture device having an image sensing element (e.g., a CCD) configured to receive light from an object and generate object-shape signals commensurate with shape of the object. The vision system also includes a processor executing software maintained on a non-transitory memory (external or resident). The processor is configured to i) receive the object-shape signals, ii) perform edge detection on the object-shape signals, and iii) generate edge-detected signals representing edges of the object. The vision system also includes one or more light-generating devices, each configured to generate light at a predetermined transmitted wavelength. The vision system also includes a plurality of artificial photoreceptors (APRs). Each APR includes a nano-dielectric core, a plurality of optical dipoles each optical dipole attached to the surface of the nano-dielectric core. Each optical dipole includes a first nano-particle configured to generate a positive voltage when receiving a light having a predetermined wavelength, and a second nano-particle configured to generate a negative voltage when receiving a light having the predetermined wavelength. One or more dielectric coatings are applied to each of the nano-dielectric cores and each of the plurality of optical dipoles. The processor controls the one or more light generating devices based on the edge-detected signals such that: i) a plurality of a first set of the first and second nano-particles of the plurality of optical dipoles cancel each other's voltages such that the plurality of APRs only generate voltages at locations corresponding to the edge-detected signals; or ii) a plurality of a second set of the first and second nano-particles of the plurality of optical dipoles generate positive and negative voltages to sequentially generate responses for biological receptors.

Those having ordinary skill in the art will recognize that numerous modifications can be made to the specific implementations described above. The implementations should not be limited to the particular limitations described. Other implementations may be possible.

1. A vision system, comprising:
an image capture system, comprising:
an image capture device having an image sensing element configured to receive light from an object and generate object-shape signals commensurate with shape of the object,
a processor executing software maintained on a non-transitory memory and configured to:
receive the object-shape signals,
perform edge detection on the object-shape signals,
generate edge-detected signals representing edges of the object, and
one or more light-generating devices, each configured to generate pulses of light at a predetermined transmitted wavelength; and
a plurality of artificial photoreceptors (APRs), each comprising:
a nano-dielectric core,
a plurality of optical dipoles each optical dipole attached to the surface of the nano-dielectric core, and each optical dipole comprising:
a first nano-particle configured to generate a positive voltage when receiving a light having a predetermined wavelength, and
a second nano-particle configured to generate a negative voltage when receiving a light having the predetermined wavelength,
wherein one or more dielectric coatings applied to each of the nano-dielectric cores and each of the plurality of optical dipoles,
wherein the processor controls the one or more light generating devices based on the edge-detected signals such that: i) a plurality of a first set of the first and second nano-particles of the plurality of optical dipoles cancel each other's voltages such that the plurality of APRs only generate voltages at locations corresponding to the edge-detected signals; or ii) a plurality of a second set of the first and second nano-particles of the plurality of optical dipoles generate positive and negative voltages to sequentially generate responses for biological receptors.

2. The vision system of claim 1, wherein the first nanoparticle of each of the plurality of optical dipoles is a gold nanoparticle.

3. The vision system of claim 2, wherein the second nanoparticle of each of the plurality of optical dipoles is a silver nanoparticle.

4. The vision system of claim 1, wherein the nano-dielectric core of each of the plurality of optical dipoles is made of a material having a dielectric constant above a dielectric threshold.

5. The vision system of claim 4, wherein the nano-dielectric core of each of the plurality of dipoles is made of BaTiO₂ or Graphene.

6. The vision system of claim 1, wherein each of the one or more dielectric coatings is made of a plasmon dielectric material.

7. The vision system of claim 1, wherein each of the one or more dielectric coatings is made of a material selected from the group consisting of polyvinylidene fluoride (PVDF), polytetrafluoroethylene (PTFE), fluorinated ethylene propylene (FEP), polyvinyl chloride (PVC), chlorinated polyvinyl chloride (CPVC), high-density polyethylene (HDPE), and combinations thereof.

8. The vision system of claim 1, wherein object-shape signals include pixel intensity values for a plurality of pixels.

9. The vision system of claim 1, wherein the processor performs edge detection based on abrupt changes in pixel intensity of the plurality of pixels.

10. The vision system of claim 1, wherein the processor performs edge detection based on one or more of object recognition, segmentation, feature extraction, or combinations thereof.

11. An artificial photoreceptors (APR), comprising:
a nano-dielectric core,
a plurality of optical dipoles each optical dipole attached to the surface of the nano-dielectric core, and each optical dipole comprising:
a first nano-particle configured to generate a positive voltage when receiving a light pulse having a predetermined wavelength, and
a second nano-particle configured to generate a negative voltage when receiving a light pulse having the predetermined wavelength,
wherein one or more dielectric coatings applied to the nano-dielectric core and each of the plurality of optical dipoles,
wherein i) a plurality of a first set of the first and second nano-particles of the plurality of optical dipoles cancel each other's voltages such that a plurality of APRs only generate voltages at locations corresponding to edge-detected signals representing edges of an object; or ii) a plurality of a second set of the first and second nano-particles of the plurality of optical dipoles generate positive and negative voltages to sequentially generate responses for biological receptors.

12. The APR of claim 11, wherein the first nanoparticle of each of the plurality of optical dipoles is a gold nanoparticle.

13. The APR of claim 12, wherein the second nanoparticle of each of the plurality of optical dipoles is a silver nanoparticle.

14. The APR of claim 11, wherein the nano-dielectric core of each of the plurality of optical dipoles is made of a material having a dielectric constant above a dielectric threshold.

15. The APR of claim 14, wherein the nano-dielectric core of each of the plurality of dipoles is made of BaTiO₂ or Graphene.

16. The APR of claim 11, wherein each of the one or more dielectric coatings is made of a plasmon dielectric material.

17. The APR of claim 11, wherein each of the one or more dielectric coatings is made of a material selected from the group consisting of polyvinylidene fluoride (PVDF), polytetrafluoroethylene (PTFE), fluorinated ethylene propylene (FEP), polyvinyl chloride (PVC), chlorinated polyvinyl chloride (CPVC), high-density polyethylene (HDPE), and combinations thereof.

18. The APR of claim 11, wherein the edge-detected signals include pixel intensity values for a plurality of pixels.

19. The APR of claim 11, wherein edge detection of the object is based on abrupt changes in pixel intensity of the plurality of pixels.

20. The APR of claim 11, wherein edge detection of the object is based on one or more of object recognition, segmentation, feature extraction, or combinations thereof.
